(19) United States
(12) Patent Application Publication
(10) Pub. No.: US 2006/0128814 A1
(43) Pub. Date: Jun. 15, 2006

(54) Photolytic cross-linkable monomers

(76) Inventors: Scott Diamond, Bala Cynwyd, PA (US); Moon Suk Kim, Daejeon (KR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/281,524

(22) Filed: Nov. 17, 2005

Related U.S. Application Data
(60) Provisional application No. 60/628,912, filed on Nov. 18, 2004.

(51) Int. Cl. A61K 31/425

(52) U.S. Cl. 514/867; 514/367

(57) ABSTRACT

Photolytic cross-linkable polymers comprises three domains, a cationic domain, a cross-linkable domain and a photolabile domain. The photolytic cross-linkable polymers according to the current invention are useful in a method to complex and compact DNA and RNA for delivery to a living cell, wherein the DNA or RNA is released by photolytic degradation of a cross-linked polymer, which encapsulates the DNA or RNA in a nanoparticle.

### RELATED APPLICATION AND CLAIM OF PRIORITY

This application claims priority under 35 U.S.C. § 119(e) to Provisional Application Ser. No. 60/628,912, which was filed on Nov. 18, 2004, the content of which is hereby incorporated by reference in its entirety.

### FIELD OF THE INVENTION

The present invention relates generally to the field of mechanisms for the delivery of biological materials to targeted sites, such as living cells. More particularly, the present invention relates to chemical compounds engineered to complex and compact biological materials for transfection to living cells and release on exposure to light of a specific wavelength.

### BACKGROUND OF THE INVENTION

Since its first use as a non-viral vector in 1995 by Boussif, Lezoualc'h et al., polyethylenimine (PEI) has been the frontrunner of cationic polymers used for gene delivery. Proc. Nat'l. Acad. Sci. USA, 92(16): 7297-301 (1995). PEI is a densely positively charged cationic polymer whose every third atom is an amine (for branched PEI the ratio of 1°/2°/3° amines is 25/50/25). Early studies focused on the physical properties of the PEI/pDNA polyplexes (such as size and zeta potential) and transfection efficiency in different cell lines as a function of polymer nitrogen to plasmid DNA (pDNA) phosphate ratio (N/P ratio) and polymer size. It was found that polyplexes made with higher molecular weight PEI (70 kDa) had a greater transfection efficiency than those made with lower molecular weight PEI (<1800 Da). Godbey, Wu et al., J. Cont. Rel. 60(2-3): 149-60. (1999). Some routes of PEI investigation have veered toward modification in order to enhance transfection efficiency and address some of the polymer's shortcomings, such as aggregation, cytotoxicity, and lack of cell selectivity. In terms of the rate-limiting barriers for non-viral gene therapy, the mechanisms of polyplex dissociation/pDNA unpackaging remain unclear and control of this process is limited.

A fine-tuned balance needs to be achieved between a polyplex that is packaged too loosely, allowing for pDNA degradation by cellular nucleases, and a polyplex that is packaged too tightly, not allowing for dissociation and transcription. Unpackaging studies with polylysine/pDNA polyplexes showed that shorter polylysines (19 residues) dissociated from pDNA faster than longer polylysines (180 residues). Schaffer, Fidelman et al., Biotech. Bioeng. 67(5): 598-606. (2000). The problem of overcoming the unpackaging barrier is two-fold, as the polymer needs to protect the pDNA and release the pDNA at a desired time/location.

Cross-linking the amine groups of polylysine with dimethyl-3,3′-dithiobispropionimidate (DTBP) resulted in polyplexes that did not aggregate and did protect the pDNA from displacement with dextran sulfate; however, preliminary in vitro studies showed an inability to transfect unless the cross-linking was first reversed with dithiothreitol (DTT). Trubetskoy, Loomis et al., Bioconjug. Chem. 10(4): 624-8. (1999). Further studies by a different group showed polylysine-DTBP complexes further stabilized with polyethylene glycol (PEG) had increased stabilization and circulation times in vivo. However, in vitro testing showed this polyplex to have poor transfection efficiency when cross-linked with DTBP. This problem was alleviated by microinjection of the cross-linked complexes directly into the cytoplasm or the nucleus; however, this method yielded the same results for uncross-linked and highly cross-linked polyplexes.

There have been several attempts at engineering pH sensitive lipids and polymers for controlled pDNA release. Polymers have been grafted with side chains that degrade rapidly at pH 7.4 and very slowly at pH 5; the polymer should remain intact in the endosomes and lysosmes to protect the pDNA from cellular nucleases, but should degrade and facilitate pDNA release once the polyplex is in the cytoplasm. Polymers have also been cross-linked with degradable moieties to enable faster degradation at pH >7 with negligible degradation at pH=5. Fusigenic peptides, such as GALA, KALA and the influenza virus HA2 N-terminal peptide, have been grafted onto lipids and polymers to facilitate in endosomal disruption. These peptides work by low pH-dependent fusion to the endosome membrane leading to membrane destabilization.

Although significant progress has been made in engineering control of pDNA release into the vectors, the level of control is still very limited. The cross-linking mechanisms discussed rely on cellular enzymes to cleave the polymer and release the pDNA, and the pH sensitive mechanisms only prevent release in the endosomes.

Photolabile protecting groups have been used extensively since their introduction in 1962 by Barltrop and Schofield, for controlled release, spatial, temporal, concentration dependent, and kinetic studies. Tet. Let. 16: 697-699 (1962). The advantage of using photolabile protecting groups is that the “caged” compounds are rendered inert until photolysis (which should liberate the compound in μs to ms time frames). The utility of such compounds has been demonstrated in mechanistic studies, such as studying inositol 1,4,5-trisphosphate's (InsP3) role in Ca²⁺ wave formation and propagation in smooth muscle cells. McCarron, MacMillan et al., J. Biol. Chem. 279(9): 8417-27 (2004). Also in drug delivery studies where targeting a very specific population of cells is of paramount importance. Perdicakis, Montgomery et al., Bioorg. Med. Chem. 13(1): 47-57 (2005). Numerous caged compounds for biological use have been synthesized, including, but not limited to, ATP and analogues, alanine, nitric oxide, nitric oxide inhibitors, receptor ligands, pDNA, Ca²⁺, and InsP3; some are even commercially available.

The potential to use this technology for gene delivery, however, has not been fully realized. Monroe et al synthesized a 1-(4,5-dimethoxy-2-nitrophenyl)diazoethane (DMNPE) caged pDNA for spatially controlled gene delivery. Monroe, McQuain, et al., J. Biol. Chem. 274(30): 20895-900 (1999). Although they saw significant decrease in the amount of pDNA that could be transcribed for the caged pDNA compared to both uncaged pDNA (where the caging group had been photocleaved) and native pDNA (where no caging groups were ever present), there was still some leakage of expression. One limitation of the approach of Monroe et al., is that the pDNA is directly modified to obtain caging. Further, the photolysis of the caged pDNA seemed to cause pDNA nicking. And finally, the caged pDNA had to be formulated with liposomes or precipiated onto gold beads into order to enter the cell.

Limited spatial targeting has been achieved by attaching receptor specific ligands to the carriers. Complexes with these ligands have been able to condense pDNA and deliver it to a targeted cell population, where it is released. However, the release mechanisms are still largely uncontrolled.

There has remained, until the present invention, a need for a novel photolabile monomer for gene delivery, which will be able to mimic many features of viral gene delivery. The monomer should be able to condense the pDNA, retain and protect the pDNA, and selectively release the pDNA into a targeted cell population. The present inventors have designed a photolabile carrier for gene delivery with three functional domains—a cationic domain to electrostatically interact with and condense the pDNA, a cross-linking domain to entrap the pDNA within the polyplex, and a photolabile domain to release the pDNA with the addition of light of an appropriate wavelength. Although the goals of spatially and temporally controlled gene delivery are similar to previous work, the methodology does not chemically alter the pDNA, but rather uses the carrier for the spatially and temporally controlled release of the pDNA.

### SUMMARY OF THE INVENTION

The present invention provides photolabile cross-linkable monomer having the general formula:
L-X-M; Formula I

wherein;

L comprises a straight chain or branched polyamine ligand;

M comprises a residue containing a cross-linkable functional moiety; and

X comprises a photolabile moiety containing an carboxylate, sulfate or phosphinate ester functional group and a nitrobenzyl functional group, wherein on exposure to light having a wavelength in the range of 300 to 450 nm, especially 365 nm, the ester functional group is cleaved by internal reaction with the nitrobenzyl functional group, thereby separating the polyamine ligand from the cross-linkable moiety.

The polyamine ligand L preferably comprises a polyalkyleneimine having a molecular weight in the range of from 500 to 25,000. More preferably the polyamine ligand L comprises polyethyleneimine.

In another embodiment, the present invention provides a supported photolabile complexing material comprising a solid support, the solid support having bonded thereto a plurality of units having the general formula
L-X—S; Formula II

wherein;

L comprises a straight chain or branched polyamine ligand;

X comprises a photolabile moiety containing an carboxylate, sulfate or phosphinate ester functional group and a nitrobenzyl functional group, wherein on exposure to light having a wavelength in the range of 300 to 450 nm, especially 365 nm, the ester functional group is cleaved by internal reaction with the nitrobenzyl functional group, thereby separating the branched ligand from the support material; and S comprises a bond to the solid support material.

The polyamine ligand L preferably comprises a polyalkyleneimine having a molecular weight in the range of from 500 to 25,000. More preferably the polyamine ligand L comprises polyethyleneimine.

In another embodiment, the present invention provides a method for delivering biological material to a target site. The method comprises forming a polyplex of the biological material with a plurality of monomer units of general Formula I as described above. The plurality cationic sites on the polyamine ligands coordinate to sites on the biological material to form a polyplex. The cross-linkable functional moieties are then cross-linked to form a nanoparticle containing the polyplex. The nanoparticle is delivered to a targeted site and exposed to light having a wavelength in the range of 300 to 450 nm, especially 365 nm, thereby cleaving the photolabile bonds in the monomer units, and releasing the biological material to the target site.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

FIG. 1. Illustrates a graph of the radii of nanoparticles produced according to the current invention.

FIG. 2. Illustrates samples of free DNA and DNA that has been complexed according to the current invention and exposed to DNase.

FIG. 3. Illustrates histograms showing the expression of enhance green fluorescence protein lipofected to dividing COS cells.

### DETAILED DESCRIPTION OF THE INVENTION

The photolytic cross-linkable monomers according to the current invention may be most generally described as being a single monomer unit having three domains: a cationic domain, a cross-linkable domain and a photolabile domain, represented by the formula:
L-X-M Formula I
where L represents the cationic domain, M represents the cross-linkable domain and X represents the photolabile domain. These novel monomers have application in the fields of biology and biochemistry as carriers for delivery of biological materials, such as DNA or RNA, to target sites, such as to living cells.

The cationic domain L may for example be a polyamine ligand having multiple cationic sites. The cross-linkable domain M comprises a residue containing a moiety capable of undergoing cross-linking reactions with chemically similar moieties on adjacent monomers. The photolabile domain will contain a functional group that is capable of undergoing internal reaction to cleave the cationic domain from the cross-linkable domain on exposure to light of an appropriate wavelength.

The photolytic cross-linkable monomers according to Formula I have application in the method of the current invention for delivery of biological materials to target sites. According to the method of the current invention, the cationic domains of a plurality of the monomers according to Formula I coordinate to anionic sites, such as phosphates, on the biological material to form a polyplex. Once the polyplex is formed, cross-linking of the cross-linkable domains is initiated, for example with ammonium persulfate, to form a nanoparticle encapsulating the biological material for delivery to a living cell or other target.

Once delivered to the target, the biological material is released from the nanoparticle by inducing photolytic degradation of the photolabile domain by application of light having the appropriate wavelength, preferably in the range of 300 to 450 nm, especially 365 nm.

According to still another embodiment of the invention, a supported photolabile complexing agent is provided. In this embodiment the complexing agent comprises two rather than three domains and is supported on an appropriate support material. The supported photolabile complexing agent may be represented generally by the formula:
L-X—S Formula II
where L and X are as defined above, and S represents a bond to a solid support material. According to this embodiment, a biological material may be immobilized by complexing with the cationic domain, and then later released by inducing photolytic degradation of the photolabile domain. Cleavage of the photolabile domain is accomplished by exposure to light having an appropriate wavelength, preferably in the range of 300 to 450 nm, especially 365 nm.

The separate embodiments of the invention will now be described with reference to specific examples.

Photolytic Cross-Linkable Monomers

The photolytic cross-linkable monomers according to the current invention comprise three separate domains: a cationic domain, a cross-linkable domain and a photolytic domain.

The cationic domain comprises a straight chain or branched polyamine ligand. The polyamine ligand may be a protein, such as polylysine, or more preferably a polyalkyleneimine, such as spermine or polyethyleneimine. The polyamine ligand will preferably have a molecular weight in the range of 500 to 25,000. When speaking of ligands such as spermine (C₁₀H₂₆N₄), which have a discrete chemical formula, the molecular weight refers to formula weight of the ligand. When speaking of polymeric materials, such as polyethyleneimine or polylysine, the molecular weight refers to the weight average molecular weight of the polymer.

The cross-linkable domain comprises any moiety that is capable of cross-linking to a similar adjacent moiety, for example in the presence of an initiator. Preferred examples include acrylate and acrylamide moieties, more preferably methacrylate or methacrylamide moeities. Preferably, the cross-linkable moiety is incorporated as the end group on a residue that is pendant from the benzyl group comprising the photolabile domain, as shown in Schemes 1 through 3.

The photolabile domain may comprise a carboxylate, phosphinate or sulfate ester group and a nitrobenzyl group, wherein the nitrobenzyl group reacts to cleave the ester on exposure to light of the appropriate wavelength. Exemplary, non-limiting structures for each embodiment of the photolabile domain are shown in Schemes 1 through 3.

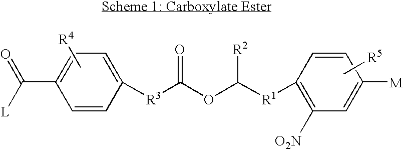

Referring to Scheme 1, M represents the residue containing the cross-linkable moiety, and L represents the polyamine ligand. R¹ and R³ are independently C₁ to C₆ alkylene or a covalent bond. Preferably, both R¹ and R³ are covalent bonds. R² is C₁ to C₆ alkyl, preferably methyl. R⁴ is selected from hydrogen, C₁ to C₆ alkyl and CO₂R⁶, wherein R⁶ is hydrogen or C₁ to C₆ alkyl. R⁵ is hydrogen, C₁ to C₆ alkyl or —OR⁷, wherein R⁷ is hydrogen or C₁ to C₆ alkyl.

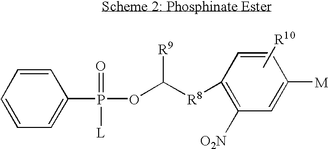

Referring to Scheme 2, M again represents the residue containing the cross-linkable moiety, and L represents the polyamine ligand. R⁸ is C₁ to C₆ alkylene or a covalent bond. Preferably, R⁸ is a covalent bond. R⁹ is C₁ to C₆ alkyl, preferably methyl, and R¹⁰ is hydrogen, C₁ to C₆ alkyl or —OR¹¹, wherein R¹¹ is hydrogen or C₁ to C₆ alkyl.

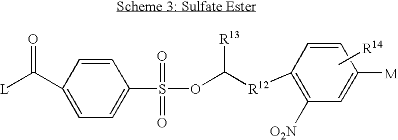

Referring to Scheme 3, M again represents the residue containing the cross-linkable moiety, and L represents the polyamine ligand. R¹² is C₁ to C₆ alkylene or a covalent bond. Preferably R¹² is a covalent bond. R¹³ is C₁ to C₆ alkyl, preferably methyl, and R¹⁴ is hydrogen, C₁ to C₆ alkyl or —OR¹⁵, wherein R¹⁵ is hydrogen or C₁ to C₆ alkyl.

Referring to Scheme 4, the photolytic cleavage of the photolabile domain is illustrated for a photolytic cross-linkable monomer according to Scheme 1.

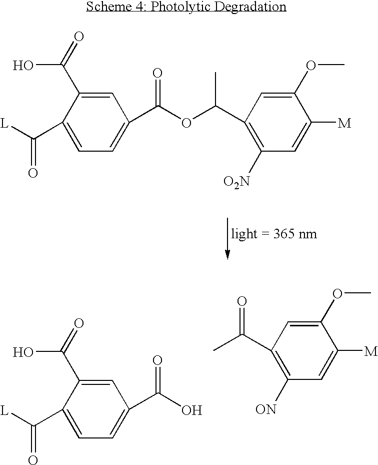

### EXAMPLES

### Synthesis of Photolytic Cross-Linkable Monomer

A photolytic cross-linkable monomer according to Scheme 1 was synthesized using the following preparation, which is illustrated in Scheme 5. Each intermediate was identified by proton NMR.

### Tert-butyl (4-acetyl-2-methoxyphenoxy)acetate

Acetovanillone (1) (3.8 g, 22.87 mmol), tert-butyl bromoacetate (4.68 g, 24.01 mmol) (Fisher Scientific), and K₂CO₃ (5.21 g, 37.70 mmol) were stirred in DMF (15 mL) at room temperature for 48 hours. The resulting solution was filtered, poured into dH₂O, and extracted with EtOAc and saturated NaCl. The combined organic layer was dried with MgSO₄ and concentrated by evaporation to yield (2): tert-butyl (4-acetyl-2-methoxyphenoxy)acetate (4.09 g, quantitative) as an off-white solid. ¹H NMR (300 MHz, CDCl₃) δ 1.47 (s, 9H), 2.56 (s, 3H), 3.94 (s, 3H), 4.66 (s, 2H), 6.77 (d, 1H), 7.53 (m, 2H).

### (4-acetyl-2-methoxy-5-nitrophenoxy)acetic acid

A solution of tert-butyl (4-acetyl-2-methoxyphenoxy)acetate (5.8 g, 20.69 mmol) in 15 mL acetic anhydride was added drop-wise to a solution of 15 mL of 70% HNO₃ and 10 mL of acetic anhydride, and stirred for 2 hours at 0° C. followed by 4 hours at room temperature. The solution was poured into dH₂O and chilled overnight to 4° C. The product was isolated by filtration, washed with water, and dried in vacuo overnight to yield of (3): (4-acetyl-2-methoxy-5-nitrophenoxy)acetic acid (2.8 g, 85% yield) as a light yellow solid. ¹H NMR (300 MHz, MeOH) δ 2.50 (s, 3H), 3.98 (s, 3H), 4.83 (s, 2H), 7.07 (s, 1H), 7.63 (s, 1H).

### N-(3-(4-acetyl-2-methoxy-5-nitrophenoxy)acetamide)propyl methacrylamide

(4-acetyl-2-methoxy-5-nitrophenoxy)acetic acid (1.2 g, 4.45 mmol), N-hydroxysuccinimide (1.2 g, 10.42 mmol), and N,N-dicyclohexylcarbodiimide (1.6 g, 7.75 mmol) in 15 mL CH₂Cl₂ (Sigma-Aldrich) were stirred under dry N₂ at room temperature to make an activated carboxylic group. After 1 hour N-(3-aminopropyl)methacrylamide hydrochloride (1.2 g, 6.71 mmol) (Polysciences, Inc., Warrington, Pa.) was added and stirred for 30 minutes. Triethylamine (0.576 g, 5.69 mmol) (Fisher Scientific) was added and the solution was stirred for 48 hours. The product was filtered, poured into dH₂O and extracted with EtOAc and saturated NaHCO₃. The combined organic layers were dried with MgSO₄ and concentrated by evaporation. The resulting product was dissolved in acetonitrile, filtered, and concentrated by evaporation to yield (4): N-(3-(4-acetyl-2-methoxy-5-nitrophenoxy)acetamide)propyl methacrylamide as a viscous yellow oil in 80% yield. ¹H NMR (300 MHz, CDCl₃) δ 1.72 (m, 2H), 1.96 (s, 3H), 2.48 (s 3H), 3.35 (2q, 4H), 4.02 (s, 3H), 4.59 (s, 2H), 5.33 (s, 1H), 5.74 (s, 1H), 6.54 (s, 1H), 6.78 (t, 1H), 7.24 (s, 1H), 7.66 (t, 1H).

### N-(3-(4-(1-hydroxylethyl)-5-nitrophenoxy)acetamide)propyl methacrylamide

NaBH₄ (0.132 g, 3.49 mmol) (Fisher Scientific) was added to N-(3-(4-acetyl-2-methoxy-5-nitrophenoxy)acetamide)propyl methacrylamide (0.71 g, 1.93 mmol) in 10 mL EtOH at 0° C. and stirred overnight, warming to room temperature. A second equivalent of NaBH₄ (0.132 g, 3.49 mmol) was added at 0° C. and stirred under dry N₂ overnight, warming to room temperature. The solution was extracted with EtOAc and NH₄Cl, and dried with MgSO₄. The product was concentrated by evaporation and purified by a silica gel column (1:1 CHCl₃:acetonitrile) to give (5): N-(3-(4-(1-hydroxylethyl)-5-nitrophenoxy)acetamide)propyl methacrylamide, a light yellow oil (0.52 g, 1.32 mmol). ¹H NMR (300 MHz, CDCl₃) δ 1.53 (d 3H), 1.72 (m 2H), 2.00 (s, 3H), 3.38 (2q, 4H), 4.00 (s, 3H), 4.56 (s, 2H), 5.33 (s, 1H), 5.54 (q, 1H), 5.75 (s, 1H), 6.65 (t, 1H), 7.27 (t, 1H), 7.40 (s, 1H), 7.60 (s, 1H).

### N-(3-(4-(1-(2,3-anhydridehydroxylethyl)-5-nitrophenoxy)acetamide)propyl methacrylamide

Trimellitic anhydride chloride (0.22 g, 1.06 mmol) and MS 4 Å molecular sieves (0.30 g) were added to a round bottom flask and purged with dry N₂ gas for 5 minutes, after which CH₂Cl₂ (6.2 mL) and anhydrous pyridine (0.12 mL, 1.57 mmol) (Sigma-Aldrich) were added. The mixture was cooled to 0° C. and a solution of N-(3-(4-(1-hydroxylethyl)-5-nitrophenoxy)acetamide)propyl methacrylamide (0.31 g, 0.792 mmol) in CH₂Cl₂ (12.4 mL) was added dropwise. The reaction was allowed to warm up to room temperature and stirred overnight. The resulting solution was filtered, poured into EtOAc, extracted with dH₂O and diluted NaHCO₃, dried with anhydrous MgSO₄, and evaporated to yield (6): N-(3-(4-(1-(2,3-anhydridehydroxylethyl)-5-nitrophenoxy)acetamide)propyl methacrylamide, a light yellow solid. ¹H NMR (300 MHz, acetone-d6): δ 1.80 (m, 2H), 1.89 (d, 3H), 1.96 (s, 3H), 3.20-3.33 (m, 4H), 4.00 (s, 3H), 4.67 (s, 2H), 5.28 (s, 1H), 5.69 (s, 1H), 6.66 (m, 1H), 7.45 (m, 1H), 7.73 (s, 1H), 8.30 (d, 1H), 8.48 (d, 1H), 8.66 (s, 1H).

### Polyethylenimine grafted with N-(3-(4-(1-(2,3-anhydridehydroxylethyl)-5-nitrophenoxy) acetamide)propyl methacrylamide (P25M)

P25M was synthesized with N-(3-(4-(1-(2,3-anhydridehydroxylethyl)-5-nitrophenoxy)acetamide)propyl methacrylamide at ratios of 1:1, 5:1 and 10:1 of N-(3-(4-(1-(2,3-anhydridehydroxylethyl)-5-nitrophenoxy)acetamide)propyl methacrylamide to PEI (referred to as P25M 1:1, P25M 5:1 and P25M 10:1, respectively). The methods for the synthesis of all three variations are the same, only the mole ratio of reactants is changed accordingly. For synthesis of P25M 1:1: a solution of polyethylenimine (MW=25,000) (1.85 g, 0.074 mmol) in 12.34 mL dH₂O/THF (v/v=40/60) under dry N₂ was added N-(3-(4-(1-(2,3-anhydridehydroxylethyl)-5-nitrophenoxy)acetamide)propyl methacrylamide (0.047 g, 0.082 mmol) in 4.94 mL THF. The reaction was stirred for 30 minutes, and the solvent was evaporated to yield (7) P25M, as a yellow oil.

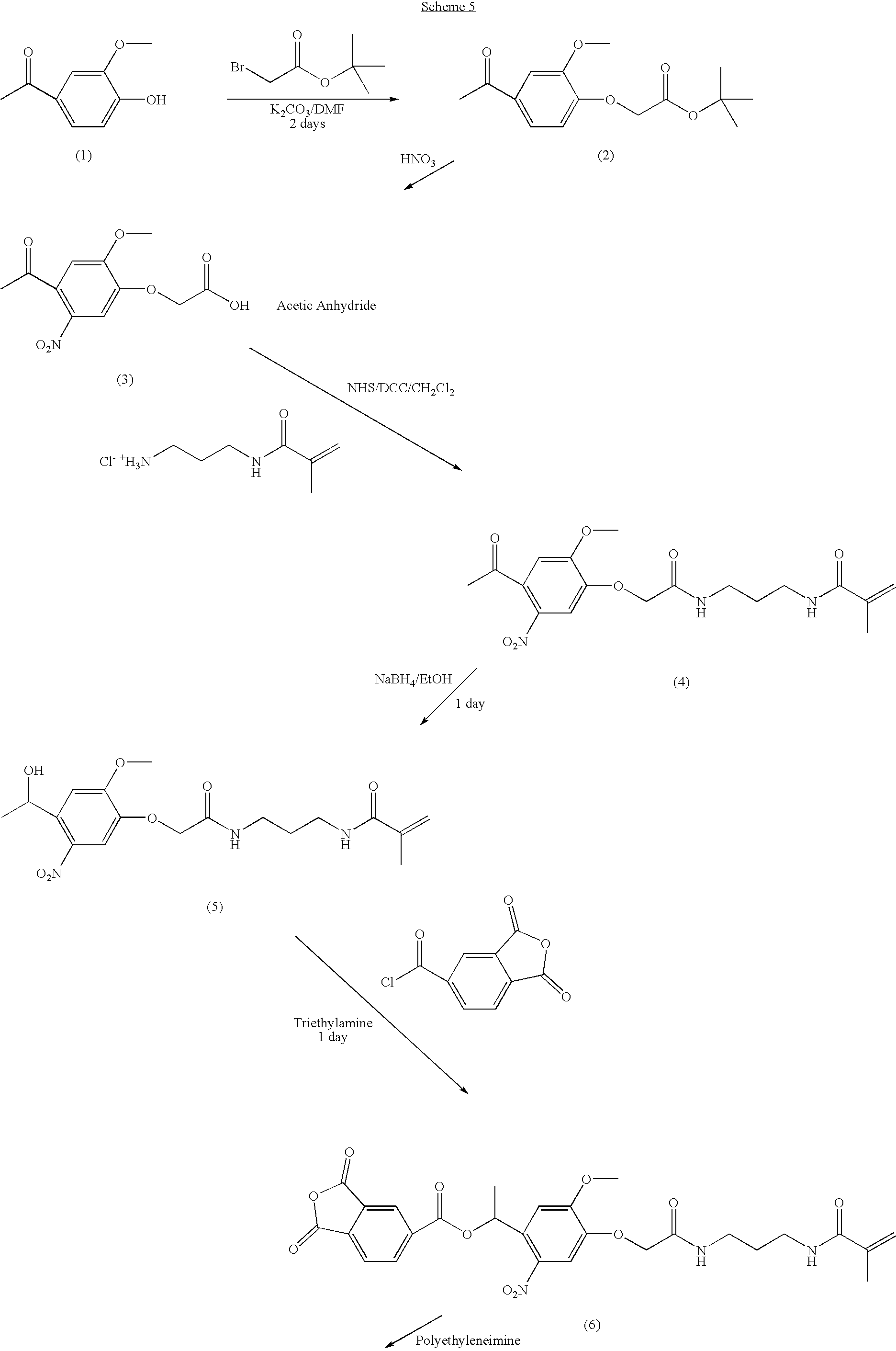

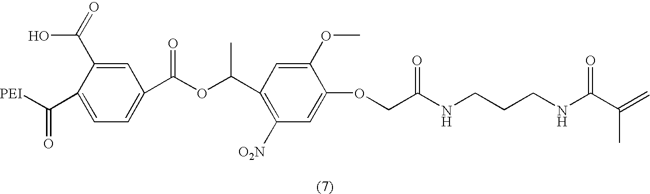

### Method for Delivery of Biological Material to a Targeted Site

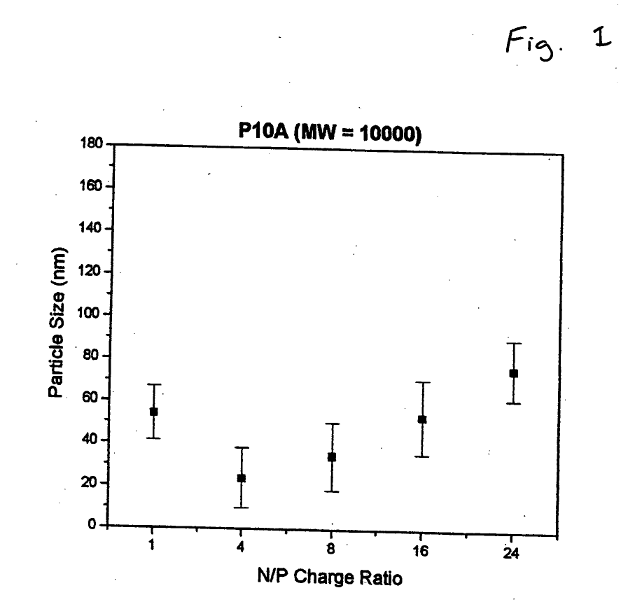

A monomer was produced according to Scheme 5 using a polyethyleneimine (PEI) having a molecular weight of 10,000, designated (P10A). P10A condensed DNA into nanoparticles with a radius of less than 80 nm. The radius of the nanoparticles was dependent upon the nitrogen/phosphate ratio (N/P) used, as shown in FIG. 1. As shown in FIG. 1 the optimal packing was observed at N/P charge ratio of 4.

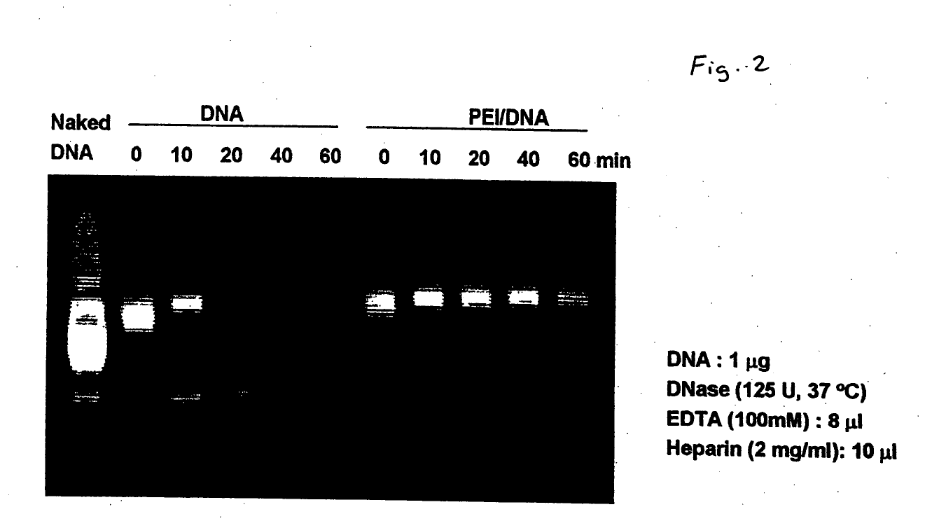

As shown in FIG. 2, condensation of DNA with P10A monomer protected DNA from DNase, whereas free DNA was rapidly degraded by added DNase. The DNA was competed off the polymer using the anionic displacer, heparin.

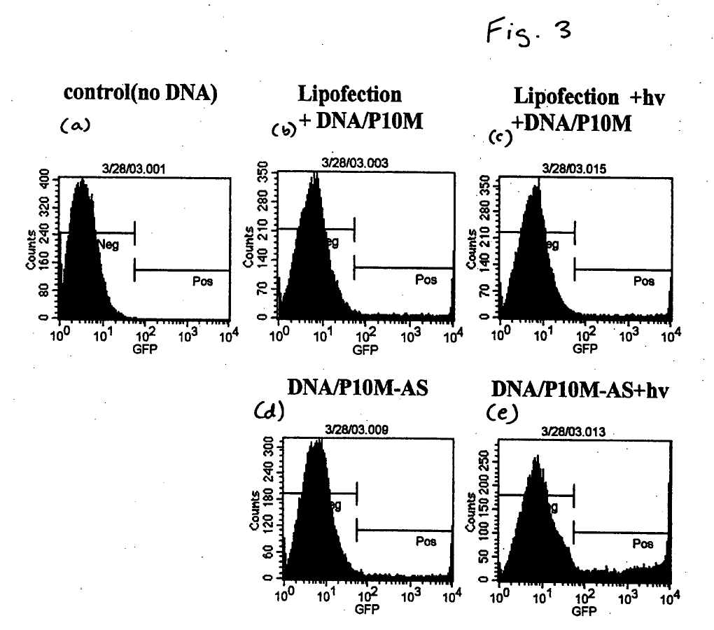

Several samples were prepared and lipofected into dividing COS cells. Ammonium persulfate (AS) initiator was used to cross-link the nanoparticle to form DNA/P10A-AS. Post-lipofection photo-irradiation was then tested for release of DNA intracellularly. Exposure to photo-irradiation at 365 nm (3.5 mW/cm²) for 10 min caused a 3-fold increase in gene expression when cells were lipofected with cross-linked polymer DNA (DNA/P10M-AS) as shown in FIG. 3(a)-(e) and Table 1. The histograms in FIG. 3(a)-(e) show the count of cells in each sample that showed expression of the lipofected plasmid DNA. The control sample, FIG. 3(a) and Table 1 top row, shows a control DNA, lipofected into dividing COS cells, which produced little fluorescence. Delivery of enhanced green fluorescence protein (EGFP) plasmid with uncross-linked monomer (P10A), which was not regulated by light, resulted in 6.29% transfection as shown by an increase in the positive response in FIG. 3(b) and Table 1 second row. Delivery of EGFP plasmid with uncross-linked monomer (P10A), which was exposed to 365 nm light (P10A+hν) showed 5.71% transfection, FIG. 3(c) and Table 1 third row. This demonstrates that the uncross-linked monomer had no effect even though the monomer can be photo-cleaved. Delivery of EGFP plasmid with cross-linked monomer (P10A), which was not regulated by light, resulted in 5.67% transfection, FIG. 3(d) and Table 1 fourth row. In contrast, the amount of transfection was dramatically increased from 5.67% to 18.44% when DNA cross-linked in the polymer (P10A-AS) was exposed to 365 nm light (P10A-AS+hν), FIG. 3(e) and Table 1 fifth row.

This experiment demonstrates that DNA was caged by the cross-linked polymer and that gene expression increased markedly when the polymer was broken down with light. EGFP was monitored by flow cytometry. The percent transfection was defined as fluorescence intensity (FI) of greater than 100. The data also indicate that DNA cross-linked within the nanoparticle was protected against lysosomal DNase. Cell viability and growth were unaffected by the 10 min light exposure at 365 nm.

TABLE 1

| Sample | % FI > 100 | Mean FI |
|---|---|---|
| Control | 0.01% | 55 |
| Lipofection | 6.29% | 3266 |
| Lipofection + light | 5.71% | 3206 |
| Cross-linked polymer | 5.67% | 3187 |
| Cross-linked polymer + | 18.44% | 3274 |
| light | | |

### Supported Photolabile Complexing Agent

The supported photolabile complexing agents according to the current invention comprise two domains: a cationic domain and a photolabile domain. The photolabile domain is bonded to a suitable solid support.

The cationic domain comprises a straight chain or branched polyamine ligand. The polyamine ligand may be a protein, such as polylysine, or a polyalkyleneimine, such as spermine or polyethyleneimine. The polyamine ligand will preferably have a molecular weight in the range of 500 to 25,000. When speaking of ligands such as spermine (C₁₀H₂₆N₄), which has a discrete chemical formula, the molecular weight refers to formula weight of the ligand. When speaking of polymeric materials, such as polyethyleneimine or polylysine, the molecular weight refers to the weight average molecular weight of the polymer.

The photolabile domain is selected from the same basic structures described for the cross-linkable monomers according to the invention, i.e. carboxylate, phosphinate or sulfate esters.

The support material can be any support known in the art. Non-limiting examples include silica, glass and polymer beads. The photolabile domain is bonded to the support by reaction with reactive groups found naturally or introduced to the surface of the support material. Non-limiting examples of reactive surface groups include amines, activated hydroxyl groups and silanols.

Scheme 5 illustrates a supported photolabile complexing agent bound to a support material through a silane moiety. Such a bond may be formed for example via hydrosilation of a surface silane, or alternatively through reaction of a silanol with active surface groups, such as chlorides or hydroxyls.

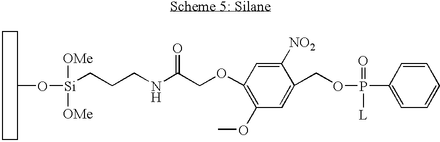

Scheme 6 illustrates a supported photolabile complexing agent bound to a support material through an amide moiety. Such a bond may be formed, for example, via reaction of a carboxylic acid or acyl halide with a surface amine.

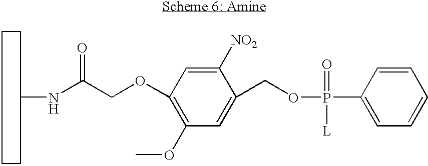

According to this embodiment, a biological material may be immobilized by complexing with the cationic domain, and then later released by inducing photolytic degradation of the photolabile domain.

The present invention provides a newly designed monomer that is cationic, cross-linkable, and photolytic, permitting the light-triggered, controlled release of a plasmid. The data presented demonstrates that using such a monomer and exposing it to light causes a 3-fold increase in transgene expression. Moreover, the designed nanoparticles yield a small packing size, DNA protection, minimal DNA leakage until photo-triggering. Accordingly, these materials allow for timed delivery of pDNA without endosomal degradation or endosomal-localized activation, potentially bypassing the deleterious endosomal toll-like receptor response. Consequently, such photo-regulated, cross-linkable reagents offer new options in the useful generation of stable DNA nanoparticles for temporally-controlled, spatial-addressed, and metered dosing of DNA for gene transfer.

The externally-controlled cleavage of covalently linked pro-drugs, proteins, or solid phase formulation vehicles offers potential advantages for controlled drug or gene delivery.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims. The full scope of the present invention will be apparent from the appended claims.

What is claimed is:

1. A photolabile cross-linkable monomer having the general formula
L-X-M;
wherein;
L comprises a straight chain or branched polyamine ligand;
M comprises a residue containing a cross-linkable moiety; and
X comprises a photolabile moiety containing an carboxylate, sulfate or phosphinate ester functional group and a nitrobenzyl functional group, wherein on exposure to light having a wavelength in the range of 300 to 450 nm, the ester functional group is cleaved by internal reaction with the nitrobenzyl functional group, thereby separating the polyamine ligand from the cross-linkable moiety.

2. The photolabile cross-linkable monomer according to claim 1, wherein
L comprises a polyalkyleneimine having a molecular weight ranging from about 500 to about 25,000.

3. The photolabile cross-linkable monomer according to claim 2, wherein
L comprises a polyethyleneimine.

4. The photolabile cross-linkable monomer according to claim 1, wherein
the cross-linkable moiety is an acrylate or acrylamide.

5. The photolabile cross-linkable monomer according to claim 1, wherein
X has the general structure; 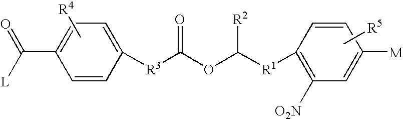
wherein
R¹ and R³ are independently C₁ to C₆ alkylene or a covalent bond;
R² is C₁ to C₆ alkyl;
R⁴ is selected from hydrogen, C₁ to C₆ alkyl and CO₂R⁶, wherein R⁶ is hydrogen or C₁ to C₆ alkyl; and
R⁵ is hydrogen, C₁ to C₆ alkyl or —OR⁷, wherein R⁷ is hydrogen or C₁ to C₆ alkyl.

6. The photolabile cross-linkable monomer according to claim 1, wherein
X has the general structure; 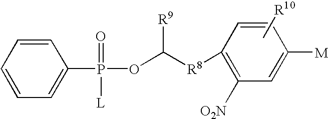
wherein
R⁸ is C₁ to C₆ alkylene or a covalent bond;
R⁹ is C₁ to C₆ alkyl; and
R¹⁰ is hydrogen, C₁ to C₆ alkyl or —OR¹¹, wherein R¹¹ is hydrogen or C₁ to C₆ alkyl.

7. The photolabile cross-linkable monomer according to claim 1, wherein
X has the general structure; 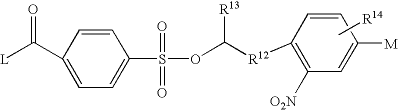
wherein
R¹² is C₁ to C₆ alkylene or a covalent bond;
R¹³ is C₁ to C₆ alkyl; and
R¹⁴ is hydrogen, C₁ to C₆ alkyl or —OR¹⁵, wherein R¹⁵ is hydrogen or C₁ to C₆ alkyl.

8. A supported photolabile complexing material comprising a solid support, the solid support having bonded thereto a plurality of units having the general formula
L-X—S;
wherein
L comprises a straight chain or branched polyamine ligand;
X comprises a photolabile moiety containing an carboxylate, sulfate or phosphinate ester functional group and a nitrobenzyl functional group, wherein on exposure to light having a wavelengths in the range of 300 to 450 nm, the ester functional group is cleaved by internal reaction with the nitrobenzyl functional group, thereby separating the branched ligand from the crosslinkable moiety; and
S comprises a bond to the solid support material.

9. The supported photolabile complexing material according to claim 8, wherein
L comprises a polyalkyleneimine having a molecular weight ranging from about 500 to about 25,000.

10. The supported photolabile complexing material according to claim 9, wherein
L comprises a polyethyleneimine.

11. The supported photolabile complexing material according to claim 8, wherein
X has the general structure; 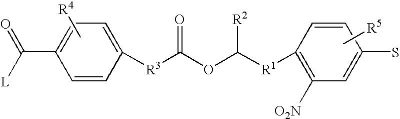
wherein
R¹ and R³ are independently C₁ to C₆ alkylene or a covalent bond;
R² is C₁ to C₆ alkyl;
R⁴ is selected from hydrogen, C₁ to C₆ alkyl and CO₂R⁶, wherein R⁶ is hydrogen or C₁ to C₆ alkyl; and
R⁵ is hydrogen, or —OR⁷, wherein R⁷ is hydrogen or C₁ to C₆ alkyl.

12. The supported photolabile complexing material according to claim 8, wherein
X has the general structure; 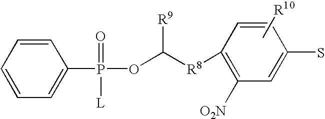
wherein
R⁸ is C₁ to C₆ alkylene or a covalent bond;
R⁹ is C₁ to C₆ alkyl; and
R¹⁰ is hydrogen, or —OR¹¹, wherein R¹¹ is hydrogen or C₁ to C₆ alkyl.

13. The supported photolabile complexing material according to claim 8, wherein
X has the general structure; 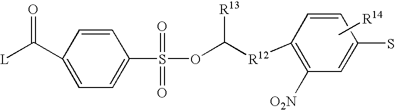
wherein
R¹² is C₁ to C₆ alkylene or a covalent bond;
R¹³ is C₁ to C₆ alkyl; and
R¹⁴ is hydrogen, or —OR¹⁵, wherein R¹⁵ is hydrogen or C₁ to C₆ alkyl.

14. A method for delivering DNA to a target site, the method comprising
forming a DNA polyplex with a plurality of monomer units having the general structure;
L-X-M;
wherein
L is a straight chain or branched polyamine ligand;
M comprises a residue containing a cross-linkable moiety; and
X comprises a photolabile moiety containing a carboxylate, sulfate or phosphinate ester functional group and a nitrobenzyl functional group, wherein on exposure to light having a wavelength in the range of 300 to 450 nm the ester functional group is cleaved by internal reaction with the nitrobenzyl functional group, thereby separating the branched ligand from the acrylate or acrylamide containing moiety;
wherein a plurality cationic sites on the polyamine ligand coordinate to sites on the DNA;
cross-linking the cross-linkable moieties to form a nanoparticle containing the DNA polyplex;
delivering the nanoparticle to a targeted site; and
exposing the nanoparticle to light having a wavelength in the range of 300 to 450 nm thereby cleaving the at least one photolabile bond in the monomer units.

15. The method according to claim 14, wherein
L is a polyalkyleneimine having a molecular weight ranging from about 500 to about 25,000.

16. The method according to claim 15, wherein
L is a polyethylenemine.

17. The method according to claim 14, wherein
the cross-linkable moiety is an acrylate or acrylamide.

18. The method according to claim 14, wherein
the crosslinking is initiated with ammonium persulfate.

19. The method according to claim 14, wherein
X has the general structure; 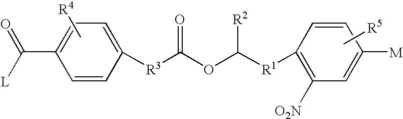
wherein
R¹ and R³ are independently C₁ to C₆ alkylene or a covalent bond;
R² is C₁ to C₆ alkyl;
R⁴ is selected from hydrogen, C₁ to C₆ alkyl and CO₂R⁶, wherein R⁶ is hydrogen or C₁ to C₆ alkyl; and
R⁵ is hydrogen, or —OR⁷, wherein R⁷ is hydrogen or C₁ to C₆ alkyl.

20. The method according to claim 14, wherein
X has the general structure; 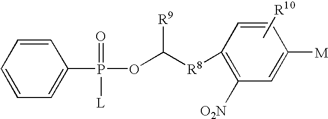
wherein
R⁸ is C₁ to C₆ alkylene or a covalent bond;
R⁹ is C₁ to C₆ alkyl; and
R¹⁰ is hydrogen, or —OR¹¹, wherein R¹¹ is hydrogen or C₁ to C₆ alkyl.

21. The method according to claim 14, wherein
X has the general structure; 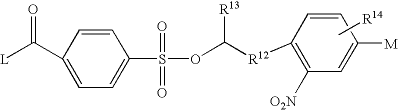
wherein
R¹² is C₁ to C₆ alkylene or a covalent bond;
R¹³ is C₁ to C₆ alkyl; and
R¹⁴ is hydrogen, or —OR¹⁵, wherein R¹⁵ is hydrogen or C₁ to C₆ alkyl.

22. The method according to claim 14, wherein the nanoparticle is exposed to light having a wavelength of 365 nm.
